(19) United States
(12) Patent Application Publication
(10) Pub. No.: US 2006/0159643 A1
(43) Pub. Date: Jul. 20, 2006

(54) Cosmetic composition comprising at least one metallic dyestuff

(76) Inventor: Isabelle Jacquier, Chevilly Larue (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/311,478

(22) Filed: Dec. 20, 2005

Related U.S. Application Data
(60) Provisional application No. 60/639,797, filed on Dec. 29, 2004.

(30) Foreign Application Priority Data
Dec. 20, 2004 (FR) 04 53081

(51) Int. Cl. A61K 8/19

(52) U.S. Cl. 424/70.7

(57) ABSTRACT

Disclosed herein is a cosmetic composition for making up or caring for keratin materials, comprising, in a cosmetically acceptable medium, a fatty phase and at least one first dyestuff comprising at least one metallic substrate coated with at least one layer of at least one mineral material. Processes for making and use are also disclosed.

This application claims benefit of U.S. Provisional Application No. 60/639,797, filed Dec. 29, 2004, the contents of which are incorporated herein by reference. This application also claims benefit of priority under 35 U.S.C. § 119 to French Patent Application No. 04 53081, filed Dec. 20, 2004, the contents of which are also incorporated herein by reference.

The present disclosure relates to a cosmetic composition for making up or caring for keratin materials such as the skin, including the scalp, of either the human face or body, and the lips, the nails and keratin fibers, such as the eyelashes, the eyebrows or the hair, comprising at least one metallic dyestuff.

The composition disclosed herein may be, for example, a makeup composition such as a loose or compacted powder, a foundation, a makeup rouge, an eyeshadow, a concealer product, a blusher, a lipstick, a lip balm, a lip gloss, a lip pencil, an eye pencil, a mascara, an eyeliner or a nail varnish, or alternatively a body makeup product or a skin coloring product.

As used herein, the term “makeup composition” means a composition intended to deposit a colored film onto keratin materials as defined above.

The use of metallic particles has already been described in various types of cosmetic makeup compositions. Thus, it is known practice to use a metal powder such as aluminium to obtain a silvery metallic effect, as proposed, for example, in International Published Application No. WO 02/03913.

However, the flakes of such a powder have problems of stability in the medium in which they are formulated and also the metallic effect and the gloss obtained are not always optimal.

The present inventor has discovered that it is possible to obtain makeup compositions with a strong metallic effect and high gloss by including in such compositions at least one metallic dyestuff. The at least one metallic dyestuff also makes it possible in certain cases to obtain a makeup of mirror type, i.e,. a makeup that not only has the color of the metal, but also gloss and a capacity to reflect the distinct elements of an object.

The strong glossy metallic effect is, for example, obtained when the content of the at least one metallic dyestuff is greater than or equal to 30% by weight, relative to the total weight of the composition.

Thus, in one embodiment, the present disclosure relates to a cosmetic composition comprising, in a cosmetically acceptable medium, a fatty phase and at least 30% of at least one dyestuff comprising at least one metallic substrate coated with at least one layer of at least one mineral material.

Another embodiment of the present disclosure provides a cosmetic process for caring for or making up keratin materials, comprising applying to said keratin materials at least one composition as defined above.

The present disclosure also relates to a process for obtaining a makeup film with a metallic and/or glossy effect on keratin materials comprising applying to said materials at least one composition as defined above

I) Dyestuff

The at least one dyestuff comprises at least one metallic substrate coated with at least one layer of at least one mineral material.

For example, the at least one metallic substrate is chosen from pure metals or alloys of pure metals (and not from metal oxides).

The at least one metallic substrate may be chosen from silver, aluminium, iron, chromium, nickel, molybdenum, gold, copper, zinc, tin, magnesium, steel, bronze and titanium, and mixtures thereof. In at least one embodiment, the at least one metallic substrate may be chosen from aluminium (for example, having an aluminium content of greater than or equal to 99%), copper (for example, having a copper content of greater than or equal to 9%) and bronze (for example, having a copper content ranging from 70% to 95% and a zinc content ranging from 5% to 30%).

In at least one embodiment, the at least one metallic substrate comprises a small content of impurities, for example less than or equal to 1%.

In at least one embodiment, the at least one metallic substrate may be in the form of platelets or leaflets.

According to one embodiment, the substrate is not treated, for example with phosphoric acid and/or boric acid compounds.

The at least one metallic substrate may be coated with at least one “coating” layer of at least one mineral material that may be, for example, chosen from metal oxides such as titanium oxide, aluminium oxide, iron oxide, cerium oxide, chromium oxide and silicon oxides, and mixtures thereof.

In at least one embodiment, the at least one metallic substrate is coated with at least one layer of silicon oxide SiO₂.

The weight ratio of the coating layer relative to the metallic substrate may range from 1 to 0.001.

The coating layer may have a thickness ranging from 5 to 500 nm, for example, ranging from 100 to 200 nm.

The metallic substrate coated with the layer of mineral material may have a mean size (D50) ranging from 1 μm to 100 μm, for example, ranging from 20 μm to 50 μm.

These dyestuffs and their preparation are described, for example, in German Patent No. DE 102 38 090.

The at least one metallic substrate coated with at least one layer of at least one metal oxide as disclosed herein may be chosen from, for example, aluminium powder coated with SiO₂ (about 10%) sold under the name VISIONAIRE BRIGHT SILVER SEA, the bronze powder (70% copper+30% zinc) coated with SiO₂ (about 2%) sold under the name VISIONAIRE BRIGHT NATURAL GOLD, the bronze power (85% copper+15% zinc) coated with SiO₂ (about 2%) sold under the name VISIONAIRE BRIGHT SUN FLOWER, the bronze powder (90% copper+10% zinc) coated with SiO₂ (about 2%) sold under the name VISIONAIRE BRIGHT SUN FLOWER, the copper powder coated with SiO₂ (about 2%) sold under the name VISIONAIRE BRIGHT CINNAMON, and the copper powder coated with SiO₂ (about 3%) sold under the name VISIONAIRE LAVA by the company Eckart.

The at least one dyestuff may be present in the composition according to the present disclosure in an amount ranging from 0.1% to 85%, for example, from 1% to 70% and further, for example, from 2% to 50% by weight, relative to the total weight of the composition.

According to one embodiment, the at least one dyestuff may be present in the presently disclosed composition in an amount of at least 30%, for example, at least 35% and further, for example, at least 40% by weight, based on the total weight of the composition.

II) Fatty Phase

The fatty phase of the composition disclosed herein comprises at least one fatty substance chosen from fatty substances that are liquid at room temperature (25° C.) and atmospheric pressure (10⁵ Pa), also known as oils; solid fatty substances or waxes; and pasty fatty substances; and mixtures thereof.

The fatty phase of the composition may be a continuous fatty phase.

a) Oils

The oils (or organic solvents) may be volatile or non-volatile, and polar or apolar.

In one embodiment, the composition disclosed herein may, for example, comprise at least one volatile oil.

As used herein, the term “volatile oil” means any non-aqueous medium capable of evaporating on contact with keratin materials in less than one hour, at room temperature and atmospheric pressure. The at least one volatile oil disclosed herein is a volatile cosmetic oil, which is liquid at room temperature, having a non-zero vapor pressure, at room temperature and atmospheric pressure, ranging from 0.13 Pa to 40 000 Pa (10⁻³ to 300 mmHg), for example, ranging from 1.3 Pa to 13 000 Pa (0.01 to 100 mmHg) and further, for example, ranging from 1.3 Pa to 1300 Pa (0.01 to 10 mmHg).

As used herein, the term “non-volatile oil” means an oil that remains on keratin materials at room temperature and atmospheric pressure for at least several hours, and that, for example, has a vapor pressure of less than 0.13 Pa (0.01 mmHg).

These non-volatile oils may be chosen from hydrocarbon-based oils or silicone oils, or mixtures thereof.

As used herein, the term “hydrocarbon-based oil” means an oil mainly comprising hydrogen and carbon atoms, and possibly oxygen, nitrogen, sulfur or phosphorus atoms. The volatile hydrocarbon-based oils may be chosen from hydrocarbon-based oils comprising from 8 to 16 carbon atoms, for example, branched C₈-C₁₆ alkanes, for instance C₈-C₁₆ isoalkanes of petroleum origin (also known as isoparaffins), for instance isododecane (also known as 2,2,4,4,6-pentamethylheptane), isodecane and isohexadecane, for example the oils sold under the trade names ISOPAR and PERMETHYL.

According to one embodiment, the fatty phase of the composition disclosed herein comprises at least one non-silicone volatile oil. For example, the non-silicone volatile oil is chosen from C₈-C₁₆ isoalkanes.

Volatile oils that may also be used include volatile silicones, for instance linear or cyclic volatile silicone oils, for example, those with a viscosity <58 centistokes (58×10⁻⁶ m²/s) and for example, comprising from 2 to 10 silicon atoms, these silicones optionally comprising alkyl or alkoxy groups comprising from 1 to 10 carbon atoms. As volatile silicone oils that may be used herein, non-limiting non-limiting mention may be made for example, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

The volatile oil may be present in the composition disclosed herein in an amount ranging from 0.1% to 95% by weight, for example, from 1% to 65% by weight and further, for example, from 2% to 50% by weight, relative to the total weight of the composition.

According to one embodiment, the fatty phase of the composition disclosed herein comprises at least one volatile oil in an amount of at least 10% by weight, relative to the total weight of the composition.

The composition may also comprise at least one non-volatile oil, chosen, for example, from non-volatile hydrocarbon-based oils and silicone oils.

Non-volatile hydrocarbon-based oils that may, for example, be mentioned include:

hydrocarbon-based oils of animal origin;

hydrocarbon-based oils of plant origin, such as triglycerides comprising fatty acid esters of glycerol, the fatty acids of which may have varied chain lengths of from C₄ to C₂₄, these chains possibly being linear or branched, and saturated or unsaturated; these oils are for example, wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, marrow oil, blackcurrant oil, evening primrose oil, millet oil, barley oil, quinoa oil, rye oil, safflower oil, candlenut oil, passionflower oil or musk rose oil; or alternatively caprylic/capric acid triglycerides, for instance those sold by the company Stéarineries Dubois or those sold under the names MIGLYOL 810, 812 and 818 by the company Dynamit Nobel;

synthetic ethers comprising from 10 to 40 carbon atoms;

linear or branched hydrocarbons of mineral or synthetic origin, such as petroleum jelly, polydecenes, hydrogenated polyisobutene such as parleam, squalane and liquid paraffins, and mixtures thereof;

synthetic esters, for instance, oils of formula R₁COOR₂ wherein R₁ is chosen from a linear or branched fatty acid residue comprising from 1 to 40 carbon atoms and R₂ is chosen from a hydrocarbon-based chain, for example, a branched chain, comprising from 1 to 40 carbon atoms, on condition that R₁+R₂≧10, for instance purcellin oil (cetostearyl octanoate), isopropyl myristate, isopropyl palmitate, C₁₂ to C₁₅ alkyl benzoate, hexyl laurate, diisopropyl adipate, isononyl isononanoate, 2-ethylhexyl palmitate, isostearyl isostearate, and alcohol or polyalcohol octanoates, decanoates or ricinoleates, for instance propylene glycol dioctanoate; hydroxylated esters, for instance isostearyl lactate or diisostearyl malate; and pentaerythritol esters;

fatty alcohols that are liquid at room temperature, with a branched and/or unsaturated carbon-based chain comprising from 12 to 26 carbon atoms, for instance octyldodecanol, isostearyl alcohol, oleyl alcohol, 2-hexyldecanol, 2-butyloctanol or 2-undecylpentadecanol;

higher fatty acids such as oleic acid, linoleic acid or linolenic acid; and mixtures thereof.

The non-volatile silicone oils that may be used in the composition disclosed herein may be non-volatile polydimethylsiloxanes (PDMS), polydimethylsiloxanes comprising alkyl or alkoxy groups, which are pendent and/or at the end of a silicone chain, these groups each comprising from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates, and mixtures thereof.

The non-volatile oils may be present in the composition disclosed herein in an amount ranging from 0.01% to 95% by weight, for example, from 0.1% to 80% by weight and further, for example, from 1% to 50% by weight (for example, 0.1% to 30%) relative to the total weight of the composition.

The fatty phase may represent from 0.01% to 98% by weight, for example, from 0.05% to 75% and further, for example, from 1% to 60% by weight, relative to the total weight of the composition.

b) Waxes

The composition disclosed herein may comprise at least one solid fatty substance or at least one wax or mixture of waxes.

The at least one wax, for example, may be a lipophilic compound, which is solid at room temperature (25° C.), with a reversible solid/liquid change of state, having a melting point of greater than or equal to 30° C. that may be up to 200° C.

By bringing the wax to the liquid state (melting), it is possible to make it miscible with the oils and to form a microscopically homogeneous mixture, but on returning the temperature of the mixture to room temperature, recrystallization of the wax in the oils of the mixture is obtained.

For example, the waxes that are suitable for the composition disclosed herein may have a melting point of greater than about 30° C., for example, greater than 45° C. and further, for example, greater than 55° C.

The melting point of the wax may be measured using a differential scanning calorimeter (DSC), for example, the calorimeter sold under the name DSC 30 by the company Mettler.

The measuring protocol is as follows:

A 15 mg sample of product placed in a crucible is subjected to a first temperature rise ranging from 0° C. to 120° C., at a heating rate of 10° C./minute, it is then cooled from 120° C. to 0° C. at a cooling rate of 10° C./minute and is finally subjected to a second temperature rise ranging from 0° C. to 120° C. at a heating rate of 5° C./minute. During the second temperature rise, the variation of the difference in power absorbed by the empty crucible and by the crucible comprising the sample of product is measured as a function of the temperature. The melting point of the compound is the temperature value corresponding to the top of the peak of the curve representing the variation in the difference in powder absorbed as a function of the temperature.

The at least one wax that may be used in the compositions disclosed herein is chosen from waxes that are solid and rigid at room temperature, of animal, plant, mineral or synthetic origin, and mixtures thereof.

The wax may also have a hardness ranging from 0.05 MPa to 30 MPa, for example, ranging from 6 MPa to 15 MPa. The hardness is determined by measuring the compression force, measured at 20° C. using the texturometer sold under the name TA-TX2i by the company Rheo, equipped with a stainless-steel cylinder 2 mm in diameter travelling at a measuring speed of 0.1 mm/s, and penetrating into the wax to a penetration depth of 0.3 mm.

The measuring protocol is as follows:

The wax is melted at a temperature equal to the melting point of the wax+20° C. The molten wax is poured into a container 30 mm in diameter and 20 mm deep. The wax is recrystallized at room temperature (25° C.) for 24 hours and is then kept at 20° C. for at least 1 hour before performing the hardness measurement. The hardness value is the maximum compression force measured divided by the surface area of the texturometer cylinder in contact with the wax.

Hydrocarbon-based waxes, for instance beeswax or lanolin wax; rice wax, Japan wax, carnauba wax, candelilla wax, microcrystalline waxes, paraffins and ozokerite; polyethylene waxes, the waxes obtained by Fisher-Tropsch synthesis and waxy copolymers, and also esters thereof, may for example, be used.

Non-limiting mention may also be made of waxes obtained by catalytic hydrogenation of animal or plant oils comprising linear or branched C₈-C₃₂ fatty chains.

Among these waxes that may, for example, be mentioned are hydrogenated jojoba oil, isomerized jojoba oil such as the trans-isomerized partially hydrogenated jojoba oil manufactured or sold by the company Desert Whale under the commercial reference Iso-Jojoba-50®, hydrogenated sunflower oil, hydrogenated castor oil, hydrogenated coconut oil, hydrogenated lanolin oil and bis(1,1,1-trimethylolpropane) tetrastearate sold under the name “Hest 2T-4S” by the company Heterene, bis(1,1,1-trimethylolpropane) tetrabehenate sold under the name “Hest 2T4B” by the company Heterene.

Non-limiting mention may also be made of silicone waxes, for instance alkyl or alkoxy dimethicones comprising from 16 to 45 carbon atoms, and fluoro waxes.

The wax obtained by hydrogenation of olive oil esterified with stearyl alcohol, sold under the name “Phytowax Olive 18 L57” or else the waxes obtained by hydrogenation of castor oil esterified with cetyl alcohol sold under the names “Phytowax ricin 16L64 and 22L73” by the company Sophim may also be used. Such waxes are described in French Patent Application No. FR-A-2 792 190.

According to at least one embodiment, the compositions disclosed herein may comprise at least one “tacky” wax, i.e. a wax with a tack of greater than or equal to 0.7 N.s and a hardness of less than or equal to 3.5 MPa, as described in European Patent Application No. EP 1 424 058, for instance the alkyl (hydroxystearyloxy)stearate waxes sold under the names “Kester Wax K 82 P®” and “Kester Wax K 80 P®” by the company Koster Keunen. The microcrystalline wax sold under the reference SP18 by the company Strahl & Pitsch, which has a hardness of about 0.46 MPa and a tack value of about 1 N.s, may also be mentioned.

The at least one wax may be present in the form of an aqueous wax microdispersion. As used herein, the term “aqueous wax microdispersion” means an aqueous dispersion of wax particles, wherein the size of the wax particles is less than or equal to about 1 μm.

Wax microdispersions are stable dispersions of colloidal wax particles, and are described for example, in “Microemulsions Theory and Practice”, L. M. Prince Ed., Academic Press (1977) pages 21-32.

The composition may have a wax content ranging from 0.1% to 50%, for example, from 1% to 40% and further, for example, from 5% to 30% by weight, relative to the total weight of the composition.

c) Pasty Fatty Substance

As used herein, the term “pasty fatty substance” means a lipophilic fatty compound comprising at a temperature of 23° C. a liquid fraction and a solid fraction.

Said pasty compound, for example, has a hardness at 20° C. ranging from 0.001 to 0.5 MPa and for example, from 0.002 to 0.4 MPa.

The hardness is measured according to a method of penetration of a probe in a sample of compound and for example using a texture analyzer (for example the TA-XT2i machine from Rheo) equipped with a stainless-steel spindle 2 mm in diameter. The hardness measurement is performed at 20° C. at the centre of five samples. The spindle is introduced into each sample at a pre-speed of 1 mm/s and then at a measuring speed of 0.1 mm/s, the penetration depth being 0.3 mm. The hardness value revealed is that of the maximum peak.

The liquid fraction of the pasty compound measured at 23° C., for example, may represent from 9% to 97% by weight of the total weight of the compound. In at least one embodiment, this liquid fraction at 23° C. represents from 15% to 85% by weight, such as from 40% to 85% by weight of the total weight of the compound. The liquid fraction by weight of the pasty compound at 23° C. is equal to the ratio of the heat of fusion consumed at 23° C. to the heat of fusion of the pasty compound.

The heat of fusion of the pasty compound is the heat consumed by the compound to change from the solid state to the liquid state. The pasty compound is said to be in the solid state when all of its mass is in solid crystalline form. The pasty compound is said to be in the liquid state when all of its mass is in liquid form.

The heat of fusion of the pasty compound is equal to the area under the curve of the thermogram obtained using a differential scanning calorimeter (DSC), such as the calorimeter sold under the name MDSC 2920 by the company TA Instrument, with a temperature rise of 5 or 10° C. per minute, according to standard ISO 11357-3: 1999. The heat of fusion of the pasty compound is the amount of energy required to make the compound change from the solid state to the liquid state. It is expressed in J/g.

The heat of fusion consumed at 23° C. is the amount of energy absorbed by the sample to change from the solid state to the state that it has at 23° C., comprising a liquid fraction and a solid fraction.

The liquid fraction of the pasty compound, measured at 32° C., represents, in at least one embodiment, 30% to 100% by weight of the compound, for example, from 80% to 100% and further, for example, from 90% to 100% by weight of the compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the heat of fusion consumed at 32° C. to the heat of fusion of the pasty compound. The heat of fusion consumed at 32° C. is calculated in the same manner as the heat of fusion consumed at 23° C.

The pasty substances may be, for example, hydrocarbon-based compounds, for instance lanolins and derivatives thereof, or alternatively polydimethylsiloxanes (PDMS).

According to one embodiment, the composition disclosed herein is in the form of a loose, compacted or cast powder, for example, comprising a silicone fatty binder.

As used herein, the expression “fatty binder” means a fatty substance or a mixture of fatty substances constituting the binder of compacted or cast powders, and also a fatty substance or mixture of fatty substances present in the loose powders for example, to increase the softness of application and to promote the adhesion to the skin.

For example, the silicone fatty binder comprises a combination of at least two components chosen from:

(a) at least one silicone oil,

(b) at least one silicone wax and/or at least one silicone pasty fatty substance,

(c) at least one silicone resin.

Consequently, another subject disclosed herein is a cosmetic composition comprising at least one dyestuff comprising at least one metallic substrate coated with at least one layer of at least one mineral material and at least one silicone fatty binder comprising a combination of at least two components chosen from:

(a) at least one silicone oil,

(b) at least one silicone wax and/or at least one silicone pasty fatty substance,

(c) at least one silicone resin.

The silicone oils may be chosen from those described above and, for example, from linear silicone oils of low viscosity (ranging from 1 to 300 cSt).

For example, the silicone oils may be linear polysiloxanes comprising (except for the end groups) units of formula (I) 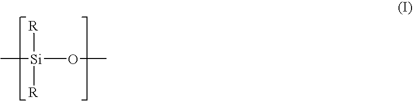
wherein each substituent R, which may be identical or different, is independently chosen from a lower alkyl group comprising from 1 to 6 carbon atoms.

The degree of polymerization (number of repeating units) of these low-viscosity polysiloxanes may range, for example, from 3 to 2000.

These low-viscosity silicone oils may be prepared according to known methods, or purchased commercially: examples that may be mentioned include the series 47 SILBIONE oil (Rhône-Poulenc), the series 200 oil (Dow Corning) and the oil SF 96 (General Electric), and mixtures thereof.

The end groups are, for example, trimethylsilyl, dimethylhydroxymethylsilyl or vinyldimethylsilyl groups.

The silicone oil may be present in an amount ranging from 12% to 98.9% by weight, relative to the total weight of the silicone binder.

The silicone waxes and/or pasty silicone fatty substances that may be used in the fatty binder are substituted polysiloxanes, for example, with a low melting point. They are, in at least one embodiment, substituted linear polysiloxanes chosen from (except for the end groups) units of formula II and III, in the respective molar proportions m and n: 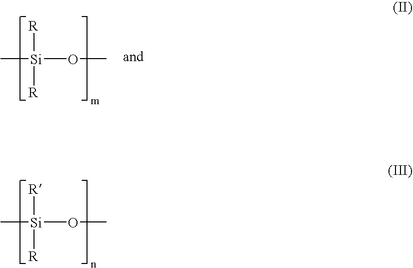
wherein:

each R, which may be identical or different, is independently chosen from a lower alkyl group comprising from 1 to 6 carbon atoms,

each R,′ which may be the same or different, is independently chosen from an optionally unsaturated (linear or branched) alkyl comprising from 6 to 30 carbon atoms, or alternatively a group —X—R″, wherein
X is chosen from:
   —O—,
   —(CH2)a-O—CO—,
   —CH2)_{b}—CO—O—,
wherein a and b, which may be identical or different, are each chosen from numbers ranging from 0 to 6, and
—R″ which may each be the same or different, independently is chosen from an optionally unsaturated alkyl group comprising 6 to 30 carbon atoms,

m is a number ranging from 0 to 400, for example from 0 to 100,

n is a number ranging from 1 to 200, for example from 1 to 100,

wherein the sum (m+n) is less than 400 and, for example, less than or equal to 100.

These silicone waxes are known or may be prepared according to known methods. Among the commercial silicone waxes of this type that may, for example, be mentioned are those sold under the names ABIL WAX 9800, 9801 or 9810 (Goldschmidt), KF910 and KF7002 (Shin-Etsu) or 176-1118-3 and 176-11481 (General Electric).

The silicone waxes that may be used may also be chosen from the compounds of formula (IV):
R₁—Si(CH₃)₂—O—[Si(R)₂—O—]_{z}—Si(CH₃)₂—R₂ (IV)
wherein:
R is defined as above,
R₁ is chosen from an alkyl group comprising from 1 to 30 carbon atoms, an alkoxy group comprising from 6 to 30 carbon atoms and a group of formula 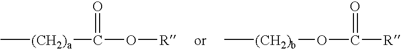
R₂ is chosen from an alkyl group of 6 to 30 carbon atoms, an alkoxy group comprising from 6 to 30 carbon atoms or a group of formula: 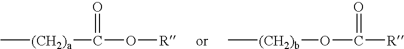
wherein, for both R₁ and R₂,

a and b, which may be identical or different, are each a number ranging from 0 to 6,

R″ is chosen from an alkyl comprising from 6 to 30 carbon atoms, and

z is an integer that may, for example, range from 1 to 100.

Among the silicone waxes of formula (IV), which are known products or which may be prepared according to known methods, non-limiting mention may be made, for example, of the following commercial products: ABIL WAX 2428, 2434 and 2440 (Goldschmidt), or VP 1622 and VP 1621 (Wacker).

The silicone wax and/or the pasty silicone fatty substance may be present in an amount ranging from 1% to 60% by weight, relative to the total weight of the silicone binder.

Silicone resins are products of hydrolysis and of polycondensation of mixtures of siloxanes of formula: (R)₃SiOCH₃ and Si(OCH₃)₄, wherein R is chosen from an alkyl group comprising from 1 to 6 carbon atoms.

These silicone resins are known or may be prepared according to known methods. Among the commercial silicone resins that may be used, for example, include those sold under the names DC 593 (Dow Corning) and SS 4230 (General Electric).

The silicone resin may be present in an amount ranging from 0.1% to 25% by weight, relative to the total weight of the silicone binder.

The silicone fatty binder may be present in an amount ranging from 0.5% to 25% by weight and, for example, from 3% to 20% by weight, relative to the total weight of the composition.

Aqueous Phase

The composition disclosed herein may comprise an aqueous phase comprising water or a mixture of water and of water-miscible solvent (miscibility in water of greater than 50% by weight at 25° C.), for instance, lower monoalcohols comprising from 1 to 5 carbon atoms such as ethanol or isopropanol, glycols comprising from 2 to 8 carbon atoms such as propylene glycol, ethylene glycol, 1,3-butylene glycol or dipropylene glycol, C₃-C₄ ketones and C₂-C₄ aldehydes, and mixtures thereof.

The aqueous phase (water and optionally the water-miscible solvent) may be present in an amount ranging from 0.1% to 95% by weight, for example, ranging from 1% to 80% by weight and further, for example, from 5% to 70% by weight, relative to the total weight of the composition.

The water and/or the water-soluble solvent(s) may be introduced in unmodified form into the formulation disclosed herein or may be incorporated by means of at least one constituent ingredient of said composition. Thus, water may for example, be introduced into the composition by means of introducing a latex or pseudolatex, i.e. an aqueous dispersion of polymer particles.

Another embodiment disclosed herein is thus a composition comprising, in a cosmetically acceptable medium, at least one aqueous phase and at least one dyestuff comprising at least one metallic substrate coated with at least one layer of at least one mineral material.

According to one embodiment, the composition disclosed herein is anhydrous. As used herein, the term “anhydrous composition” means a composition comprising an aqueous phase as defined above in a proportion of less than or equal to 10%, for example, less than or equal to 5% and further, for example, less than or equal to 3%, or even free of water.

The composition disclosed herein comprises at least one cosmetically acceptable medium, i.e. a medium that is compatible with keratin materials (acceptable tolerance, toxicology and feel).

The composition disclosed herein may also comprise a film-forming polymer.

As used herein, the term “film-forming polymer” means a polymer capable of forming, by itself or in the presence of an auxiliary film-forming agent, a continuous film that adheres to a support and for example, to keratin materials.

Among the film-forming polymers that may be used, non-limiting mention may be made of synthetic polymers, of free-radical type or of polycondensate type, and polymers of natural origin, and mixtures thereof. Film-forming polymers that may be mentioned, for example, include acrylic polymers, polyurethanes, polyesters, polyamides, polyureas and cellulose-based polymers other than liposoluble modified cellulose derivatives.

The film-forming polymers may be soluble or dispersible in a liquid fatty phase, which may be the liquid fatty phase of the composition. They may also be chosen from film-forming polymers that are water-soluble or dispersible in an aqueous phase (also known as latices).

The film-forming polymer may be present in an amount ranging from 0.1% to 30% by weight and further, for example, from 0.5% to 15% by weight of solids, relative to the total weight of the composition.

Particulate Phase

The composition disclosed herein may comprise, besides the dyestuff described above, at least one additional dyestuff chosen from water-soluble dyes, and pulverulent dyestuffs, for instance pigments, nacres and flakes that are well known to those skilled in the art, which form the “particulate” phase of the composition disclosed herein. The at least one additional dyestuff may be present in the composition in an amount ranging from 0.01% to 85% by weight and further, for example, from 0.05% to 70% by weight, relative to the total weight of the composition.

As used herein, the term “pigments” means white or colored, mineral or organic particles of any shape, which are insoluble in the physiological medium, and which are intended to color the composition.

As used herein, the term “nacres” means iridescent particles of any shape, for example, produced in the shell of certain molluscs or alternatively synthesized.

The pigments may be white or colored, and mineral and/or organic. Among the mineral pigments that may be mentioned, for example, are titanium dioxide, optionally surface-treated, zirconium oxide or cerium oxide, and also zinc oxide, iron oxide (black, yellow or red) or chromium oxide, manganese violet, ultramarine blue, chromium hydrate and ferric blue, metal powders, for instance aluminium powder or copper powder, and ultramarines.

Among the organic pigments that may be mentioned, for example, are carbon black, pigments of D&C type, and lakes based on cochineal carmine or on barium, strontium, calcium or aluminium.

Non-limiting mention may also be made of effect pigments such as particles comprising a natural or synthetic, organic or mineral substrate, for example glass, acrylic resins, polyester, polyurethane, polyethylene terephthalate, ceramics or aluminas, said substrate possibly being coated with metallic substances, for instance, aluminium, gold, silver, platinum, copper or bronze, or metal oxides, for instance, titanium dioxide, iron oxide or chromium oxide, and mixtures thereof.

The nacreous pigments may be chosen from white nacreous pigments such as mica coated with titanium or with bismuth oxychloride, colored nacreous pigments such as titanium mica coated with iron oxides, titanium mica coated for example, with ferric blue or with chromium oxide, titanium mica coated with an organic pigment of the abovementioned type, and also nacreous pigments based on bismuth oxychloride. Interference pigments, for example, comprising liquid crystals or multilayers, may also be used.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto. The water-soluble dyes are, for example, beetroot juice, methylene blue, the disodium salt of ponceau, the disodium salt of alizarin green, quinoline yellow, the trisodium salt of amaranth, the disodium salt of tartrazine, the monosodium salt of rhodamine, the disodium salt of fuchsin, and xanthophyll.

The composition disclosed herein may comprise at least one filler, for example, in an amount ranging from 0.01% to 60% by weight and further, for example, ranging from 1% to 50% by weight, relative to the total weight of the composition. As used herein, the term “fillers” means white or colorless, mineral or synthetic particles of any form, which are insoluble in the medium of the composition irrespective of the temperature at which the composition is manufactured. These fillers serve for example, to modify the rheology or the texture of the composition.

The fillers may be, for example, mineral or organic of any form, platelet-shaped, spherical or oblong, irrespective of the crystallographic form (for example lamellar, cubic, hexagonal, orthorhombic, etc.). Non-limiting mention may be made of talc, mica, silica, kaolin, polyamide (Nylon®) powder (Orgasol® from Atochem), poly-β-alanine powder and polyethylene powder, powders of tetrafluoroethylene polymers (Teflon®), lauroyllysine, starch, boron nitride, expanded hollow polymer microspheres such as those of polyvinylidene chloride/acrylonitrile, for instance Expancel® (Nobel Industrie) or of acrylic acid copolymers (Polytrap® from the company Dow Corning) and silicone resin microbeads (for example Tospearls® from Toshiba), polymethyl methacrylate particles (Micropearl M100 from Matsumoto Yushi, Covabead LH 85 from LCW or Jurymer MB1 from Nihon Junyaku), polyurethane particles, which are for example, spherical (Plastic Powder D-400, Plastic Powder D-800 and Plastic Powder CS-400 from Toshiki), elastomeric polyorganosiloxane particles, precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids comprising from 8 to 22 carbon atoms and, for example, from 12 to 18 carbon atoms, for example, zinc stearate, magnesium stearate or lithium stearate, zinc laurate or magnesium myristate.

According to one embodiment, the composition disclosed herein comprises at least one mineral filler with a refractive index ranging from 1.50 to 1.60 and a mean size (D50) of greater than or equal to 10 μm, for example, greater than or equal to 12 μm and further, for example, greater than or equal to 14 μm. The mean sizes are measured using laser-scattering granulometers such as the machine sold under the name “Mastersizer 2000” by the company Malvern.

Such a filler has a certain level of transparency, which does not have a negative effect (for example a matting effect) on the gloss and the metallic effect of the composition.

Consequently, according to another embodiment, disclosed herein is a cosmetic composition comprising, in a cosmetically acceptable medium, a fatty phase comprising at least one dyestuff comprising at least one metallic substrate coated with at least one layer of at least one mineral material and at least one mineral filler with a refractive index ranging from 1.50 to 1.60 and a mean size (D50) of greater than or equal to 10 μm, for example, greater than or equal to 12 μm and further, for example, greater than or equal to 14 μm.

Such a filler is for example talc in platelet form, with a mean size (D50) of about 14 μm, and for example, the product sold by the company Luzenac under the reference LUZENAC 00.

In another embodiment, disclosed herein is a cosmetic composition comprising, in a cosmetically acceptable medium, at least one fatty phase comprising at least one pigment and talc.

The compositions disclosed herein may also comprise ingredients commonly used in cosmetics, such as vitamins, thickeners, hydrophilic or lipophilic gelling agents, emulsifiers, trace elements, softeners, sequestrants, fragrances, acidifying or basifying agents, preserving agents, sunscreens, surfactants, antioxidants and fibers, or mixtures thereof.

The compositions disclosed herein may for example, be in the form of a suspension, a dispersion, a solution, a gel, an emulsion, for example, an oil-in-water (O/W) emulsion, a water-in-oil (W/O) or a multiple emulsion (W/O/W, polyol/O/W or O/W/O), or in the form of a cream, a paste, a mousse, a dispersion of vesicles, for example, of ionic or nonionic lipids, a two-phase or multi-phase lotion, a powder or a paste, for example, a soft paste. The composition may be anhydrous: for example, it may be an anhydrous paste or stick.

In at least one embodiment, the composition is a leave-on composition.

The compositions disclosed herein may be in the form of a makeup composition, for example, complexion products (foundations), makeup rouges, eyeshadows, sticks of lipstick, concealer products, blushers, mascaras, eyeliners, eyebrow makeup products, lip pencils, eye pencils, nail products such as nail varnishes, body makeup products or hair makeup products (hair mascara or hair lacquer). They may be in the form of a cast product, for example in the form of a stick or wand, in the form of a soft paste in a hot-water sachet, or in the form of a dish that may be used by direct contact or with a sponge, and also in the form of a soft paste or a gel, or a more or less fluid cream.

A person skilled in the art may select the appropriate galenical form, and also the method for its preparation, on the basis of his general knowledge, taking into account firstly the nature of the constituents used, for example, their solubility in the support, and secondly the intended use of the composition.

According to one embodiment, the composition disclosed herein is for coating keratin fibers (such as the eyelashes, the eyebrows or the hair).

According to another embodiment, the composition is an eyeshadow.

A subject of the present disclosure is also a cosmetic assembly comprising:

a container delimiting at least one compartment, said container being closed by means of a closing member; and

a composition as described above, placed inside said compartment.

The container may be in any useful form. It may, for example, be in the form of a bottle, a tube, a jar, a case, a box, a sachet or a carton.

The closing member may be in the form of a removable stopper, a lid, a cap, a tear-off strip or a capsule, for example, of the type comprising a body attached to the container and a cover cap articulated on the body. It may also be in the form of a member for selectively closing the container, for example, a pump, a valve or a flap valve.

The container may be combined with an applicator, for example, in the form of a brush comprising an arrangement of bristles maintained by a twisted wire. Such a twisted brush is described for example, in U.S. Patent No. U.S. Pat. No. 4,887,622. It may also be in the form of a comb comprising a plurality of application members, obtained for example, by molding. Such combs are described, for example, in French Patent No. FR 2 796 529. The applicator may be in the form of a fine brush, as described, for example, in French Patent No. FR 2 722 380. The applicator may be in the form of a block of foam or of elastomer, a felt or a spatula. The applicator may be free (tuft or sponge) or securely fastened to a rod borne by the closing member, as described, for example, in U.S. Pat. No. 5,492,426. The applicator may be securely fastened to the container, as described, for example, in French Patent No. FR 2 761 959.

The closing member may be coupled to the container by screwing. Alternatively, the coupling between the closing member and the container is done other than by screwing, for example, via a bayonet mechanism, by click-fastening, gripping, welding, bonding or by magnetic attraction. As used herein, the term “click-fastening” for example means any system involving the crossing of a bead or cord of material by elastic deformation of a portion, for example, of the closing member, followed by return to the elastically unconstrained position of said portion after the crossing of the bead or cord.

The container may be at least partially made of thermoplastic material. Examples of thermoplastic materials that may be mentioned include polypropylene or polyethylene.

Alternatively, the container is made of non-thermoplastic material, for example, glass or metal (or alloy).

The container may have rigid walls or deformable walls, for example, in the form of a tube or a tubular bottle.

The container may comprise means for distributing or facilitating the distribution of the composition. By way of example, the container may have deformable walls so as to cause the composition to exit in response to a positive pressure inside the container, this positive pressure being caused by elastic (or non-elastic) squeezing of the walls of the container. Alternatively, for example, when the product is in the form of a stick, the product may be driven out by a piston mechanism. Still in the case of a stick, for example, of makeup product (lipstick, foundation, etc.), the container may comprise a mechanism, for example, a rack mechanism, a threaded-rod mechanism or a helical groove mechanism, and may be capable of moving a stick in the direction of said aperture. Such a mechanism is described, for example, in French Patent No. FR 2 806 273 or in French Patent No. FR 2 775 566. Such a mechanism for a liquid product is described in French Patent No. FR 2 727 609.

The container may comprise a carton with a base delimiting at least one housing comprising the composition, and a lid, for example, articulated on the base, and capable of at least partially covering said base. Such a carton is described, for example, in International Published Application No. WO 03/018423 or in French Patent No. FR 2 791 042.

The container may be, for example, equipped with a drainer arranged in the region of the aperture of the container. Such a drainer makes it possible to wipe the applicator and possibly the rod to which it may be securely fastened. Such a drainer is described, for example, in French Patent No. FR 2 792 618.

The composition may be at atmospheric pressure inside the container (at room temperature) or pressurized, for example, by means of a propellant gas (aerosol). In the latter case, the container is equipped with a valve (of the type used for aerosols).

The content of the patents or patent applications mentioned above is incorporated by reference into the present patent application.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the embodiments disclosed herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosed embodiments are approximations, unless otherwise indicated the numerical values set forth in the specific examples are reported as precisely as possible. Any numerical value, however, inherently contain certain errors necessarily resulting from the standard deviation found in their respective testing measurements.

The embodiments disclosed herein are illustrated in greater detail by the examples described below.

Unless otherwise indicated, the amounts are expressed in grams.

EXAMPLE 1
Eyeshadow

| | |
|---|---|
| Rose Helindone | 0.9% |
| Hydrogenated polydecene (MW = 549) | 6% |
| Magnesium stearate | 4% |
| Yellow iron oxide | 1.5% |
| SiO₂-coated aluminium powder | 50% |
| (Visionaire Bright Silver Sea from Eckart) | |
| Glyceryl triisostearate | 6% |
| Talc | 31.4% |
| Preserving agent | 0.2% |

EXAMPLE 2
Eyeliner

| | |
|---|---|
| Oxyethenylated (4 EO) trilauryl phosphate | 2% |
| Xanthan gum | 0.3% |
| Propylene glycol | 15% |
| Diglycol/cyclohexanedimethanol/isophthalates/ | 5% |
| sulfoisophthalates copolymer (Eastman AQ | |
| 55S from Eastman) | |
| Crosslinked ethyl acrylate/methyl methacrylate copolymer | 12% |
| (80/20), as an aqueous 50% dispersion (Daitosol 5000AD | |
| from Daito Kasei) | |
| SiO₂-coated aluminium powder | 20% |
| (Visionaire Bright Silver Sea from Eckart) | |
| Preserving agents | 0.70% |
| Ethanol | 2.5% |
| Water | qs 100% |

EXAMPLE 3
Mascara

| | |
|---|---|
| Sodium hydroxide | 0.02% |
| Hydroxyethylcellulose | 1.9% |
| Mixture of polydimethylsiloxane and of hydrated silica | 0.19% |
| Propylene glycol | 5% |
| Diglycol/cyclohexanedimethanol/isophthalates/ | 19% |
| sulfoisophthalates copolymer (Eastman AQ 55S from | |
| Eastman) | |
| Microdispersion of carnauba wax | 33.46% |
| SiO₂-coated aluminium powder | 7% |
| (Visionaire Bright Silver Sea from Eckart) | |
| Preserving agent | qs |
| Ethanol | 4.65% |
| Water | qs 100% |

EXAMPLE 4
Mascara

| | |
|---|---|
| Hydroxyethylcellulose quaternized with 2,3- | 0.1% |
| epoxypropyltrimethylammonium chloride | |
| Polyethylene glycol stearate (40 EO) | 0.5% |
| Hydroxyethylcellulose | 0.89% |
| Non-stabilized sodium polymethacrylate at 25% in water | 1% |
| (Darvan 7 from Vanderbilt) | |
| Gum arabic | 3.4% |
| Beeswax | 4.37% |
| Paraffin wax | 13.82% |
| Carnauba wax | 3.45% |
| Hydrogenated jojoba oil | 0.2% |
| Hydrogenated palm oil | 0.2% |
| Mixture of polydimethylsiloxane and of hydrated silica | 0.13% |
| Black iron oxide | 0.3% |
| Ultramarine blue | 4.2% |
| SiO₂-coated aluminium powder | 2.5% |
| (Visionaire Bright Silver Sea from Eckart) | |
| Stearic acid | 6.6% |
| Triethanolamine | 2.4% |
| D-Panthenol | 0.01% |
| Preserving agents | 0.4% |
| 2-Amino-2-methyl-1,3-propanediol | 0.8% |
| Water | qs 100 |

EXAMPLE 5
Eyeshadow

| | |
|---|---|
| Polydimethylsiloxane | 0.58% |
| Phenyltrimethylsiloxytrisiloxane (DC 556 from Dow Corning) | 6.57% |
| Polydimethylsiloxane/trimethyl siloxysilicate mixture (DC 593 | 0.17% |
| Fluid from Dow Corning) | |
| Polymethylcetyldimethylsiloxane (Abil Wax 9801 from | 0.1% |
| Goldschmidt) | |
| Glyceryl triisostearate | 6.57% |
| Synthetic sericite (fluorophlogopite) | 4.28% |
| Talc | 24.18% |
| Magnesium stearate | 4.92% |
| Nylon-12 powder | 4.28% |
| SiO₂-coated aluminium powder | 40% |
| (Visionaire Bright Silver Sea from Eckart) | |
| Zinc oxide | 2.14% |
| Titanium oxide | 1.28% |
| Mica-titanium oxide | 4.28% |
| Preserving agent | 0.6% |

EXAMPLE 6
Eyeshadow

| | |
|---|---|
| Xanthan gum | 0.30% |
| Glycerol | 5% |
| Synthetic laponite | 3.0% |
| Propylene glycol | 7% |
| Partial benzoate ester of alkoxylated polydimethylsiloxane | 3% |
| (Finsolv SLB-101 from Finetex) | |
| SiO₂-coated aluminium powder | 9.2% |
| (Visionaire Bright Silver Sea from Eckart) | |
| Black iron oxide | 0.8% |
| Preserving agent | qs |
| Ethanol | 10% |
| Water | qs 100% |

EXAMPLE 7
Eyeshadow

| | |
|---|---|
| Distearyldimethylammonium-modified hectorite | 3.88% |
| Lauric/palmitic/cetylic/stearic acid triglycerides (Softisan 100 | 4.66% |
| from Sasol) | |
| Propylene carbonate | 1.26% |
| Beeswax | 5.58% |
| Paraffin wax | 2.80% |
| N-Lauroyl-L-lysine | 4.41% |
| Talc | 26.92% |
| SiO₂-coated copper powder | 11.76% |
| (Visionaire Bright Cinnamon from Eckart) | |
| Aluminium lake of cochineal carmine | 1.62% |
| Ultramarine blue | 1.62% |
| Preserving agent | 0.17% |
| Isododecane | qs 100% |

EXAMPLE 8
Loose powder

| | |
|---|---|
| Polydimethylsiloxane (DC 200 10 cSt from Dow Corning) | 2.716% |
| Polydimethylsiloxane/trimethyl siloxysilicate mixture (DC 593 | 0.792% |
| Fluid from Dow Corning) | |
| Polymethylcetyldimethylsiloxane (MW: 900 - viscosity: | 0.492% |
| 15-25 cSt) (Abil Wax 9801 from Goldschmidt) | |
| Preserving agents | 0.5% |
| Titanium oxide | 1.5% |
| Black iron oxide | 0.5% |
| Alumina-titanium oxide (Xirona Silver from Merck) | 50% |
| SiO₂-coated aluminium powder | 10% |
| (Visionaire Bright Silver Sea from Eckart) | |
| Talc | 33.5% |

EXAMPLE 9
Compact powder

| | |
|---|---|
| Polydimethylsiloxane (DC 200 10 cSt from Dow Corning) | 6.29% |
| Polydimethylsiloxane/trimethyl siloxysilicate mixture (DC 593 | 1.98% |
| Fluid from Dow Corning) | |
| Polymethylcetyldimethylsiloxane (MW: 900 - viscosity: | 1.23% |
| 15-25 cSt) (Abil Wax 9801 from Goldschmidt) | |
| Triisocetyl citrate | 0.5% |
| Isocetyl stearate | 2% |
| Preserving agents | 0.6% |
| Mica | 15% |
| SiO₂-coated bronze (90% copper and 10% zinc) powder | 10% |
| (Visionaire Bright Honey from Eckart) | |
| SiO₂-coated copper powder (Visionaire Bright Cinnamon from | 10% |
| Eckart) | |
| Yellow iron oxide | 0.5% |
| Brown, yellow iron oxide (75/25) | 0.8% |
| Black iron oxide | 0.1% |
| Zinc laurate | 3% |
| Nylon-12 powder | 5% |
| Talc | 4.3% |

EXAMPLE 10
Compact powder

| | |
|---|---|
| Isopropyl myristate | 0.22% |
| Castor oil | 0.33% |
| Liquid petroleum jelly | 1.68% |
| Lanolin oil | 0.17% |
| Petroleum jelly | 0.31% |
| Carnauba wax microbeads | 4% |
| Polyethylene glycol monostearate (8 EO) | 0.04% |
| Polyglyceryl distearate | 0.06% |
| Stearic acid | 0.03% |
| Glycerol | 0.15% |
| Preserving agents | 0.5% |
| Mica | 30% |
| Hollow polymethyl methacrylate microspheres (Covabeads LH | 5% |
| 85 from LCW) | |
| SiO₂-coated copper powder (Visionaire Bright Cinnamon from | 40% |
| Eckart) | |
| Yellow iron oxide | 0.7% |
| Brown, yellow iron oxide (75/25) | 1.1% |
| Black iron oxide | 0.1% |
| Zinc laurate | 2% |
| Nylon-12 powder | 5% |
| Talc | 8.6% |

What is claimed is:

1. A cosmetic composition comprising, in a cosmetically acceptable medium, a fatty phase and at least 30% of at least one dyestuff comprising at least one metallic substrate coated with at least one layer of at least one mineral material.

2. A composition according to claim 1, wherein the composition comprises at least 40% of said at least one dyestuff.

3. A cosmetic composition comprising, in a cosmetically acceptable medium, a fatty phase comprising at least one volatile oil present in an amount of at least 10% by weight, relative to the total weight of the composition and at least one dyestuff comprising at least one metallic substrate coated with at least one layer of at least one mineral material.

4. A cosmetic composition comprising, in a cosmetically acceptable medium, a fatty phase comprising at least one non-volatile silicone oil and at least one dyestuff comprising at least one metallic substrate coated with at least one layer of at least one mineral material.

5. A cosmetic composition comprising, in a cosmetically acceptable medium, a fatty phase comprising at least one dyestuff comprising at least one metallic substrate coated with at least one layer of at least one mineral material, said substrate being non treated with a phosphoric acid compound, and talc.

6. A cosmetic composition comprising, in a cosmetically acceptable medium, a fatty phase comprising at least one dyestuff comprising at least one metallic substrate coated with at least one layer of at least one mineral material and at least one mineral filler with a refractive index ranging from 1.50 to 1.60 and a mean size (D50) of greater than or equal to 10 μm.

7. A cosmetic composition according to claim 1, wherein the fatty phase comprises at least one fatty substance chosen from liquid fatty substances or oils, solid fatty substances or waxes, and pasty fatty substances, and mixtures thereof.

8. A composition according to claim 7, wherein the oils are chosen from hydrocarbon-based oils and silicone oils, which are volatile or non-volatile, or mixtures thereof.

9. A composition according to claim 7, wherein the oil is present in an amount ranging from 0.1% to 95% by weight, relative to the total weight of the composition.

10. A composition according to claim 9, wherein the oil is present in an amount ranging from 2% to 50% by weight, relative to the total weight of the composition.

11. A composition according to claim 1, wherein the composition comprises at least one wax.

12. A composition according to claim 11, wherein the wax is present in an amount ranging from 0.1% to 50% by weight, relative to the total weight of the composition.

13. A composition according to claim 12, wherein the wax is present in an amount ranging from 5% to 20% by weight, relative to the total weight of the composition.

14. A cosmetic composition comprising at least one dyestuff comprising at least one metallic substrate coated with at least one layer of at least one mineral material and at least one silicone fatty binder comprising at least two components chosen from:
(a) a silicone oil,
(b) a silicone wax and/or a pasty silicone fatty substance, and
(c) a silicone resin.

15. A composition according to claim 14, wherein the at least one silicone fatty binder is present in an amount ranging from 0.5% to 25% by weight, relative to the total weight of the composition.

16. A composition according to claim 15, wherein the at least one silicone fatty binder is present in an amount ranging from 3% to 20% by weight, relative to the total weight of the composition.

17. A composition according to claim 1, wherein the composition comprises an aqueous phase.

18. A cosmetic composition comprising, in a cosmetically acceptable medium, an aqueous phase and at least one dyestuff comprising at least one metallic substrate coated with at least one layer of at least one mineral material.

19. A composition according to claim 17, wherein the aqueous phase is present in an amount ranging from 0.1% to 95% by weight, relative to the total weight of the composition.

20. A composition according to claim 19, wherein the aqueous phase is present in an amount ranging from 5% to 70% by weight, relative to the total weight of the composition.

21. A composition according to claim 1, wherein the at least one metallic substrate is chosen from silver, titanium, aluminium, iron, chromium, nickel, molybdenum, gold, copper, zinc, tin, magnesium, steel, bronze and titanium, and mixtures thereof.

22. A composition according to claim 1, wherein the at least one metallic substrate is in the form of platelets.

23. A composition according to claim 1, wherein the at least one mineral material is chosen from metal oxides.

24. A composition according to claim 23, wherein the metal oxides are chosen from titanium oxide, aluminium oxide, iron oxide, cerium oxide, chromium oxide and silicon oxides, and mixtures thereof.

25. A composition according to claim 1, wherein the at least one metallic substrate is coated with at least one layer of silicon oxide (SiO₂).

26. A composition according to claim 2, wherein the at least one dyestuff is present in an amount ranging from 0.1% to 85% by weight, relative to the total weight of the composition.

27. A composition according to claim 26, wherein the at least one dyestuff is present in an amount ranging from 2% to 50% by weight, relative to the total weight of the composition.

28. A composition according to claim 1, wherein the composition comprises at least one film-forming polymer chosen from synthetic polymers, of free-radical type or of polycondensate type, and polymers of natural origin, and mixtures thereof.

29. A composition according to claim 28, wherein the at least one film-forming polymer is chosen from acrylic polymers, polyurethanes, polyesters, polyamides, polyureas, cellulose-based polymers other than liposoluble modified cellulose derivatives, and mixtures thereof.

30. A composition according to claim 28, wherein the at least one film-forming polymer is present in an amount ranging from 0.1% to 30% by weight, relative to the total weight of the composition.

31. A composition according to claim 30, wherein the at least one film-forming polymer is present in an amount ranging from 0.5% to 15% by weight, relative to the total weight of the composition.

32. A cosmetic composition according to claim 1, wherein the composition also comprises at least one additional dyestuff chosen from water-soluble dyes and pulverulent dye stuffs.

33. A composition according to claim 32, wherein the at least one additional dyestuff is present in an amount ranging from 0.01% to 85% by weight, relative to the total weight of the composition.

34. A composition according to claim 33, wherein the at least one additional dyestuff is present in an amount ranging from 0.05% to 70% by weight, relative to the total weight of the composition.

35. A composition according to claim 1, wherein the composition comprises at least one filler.

36. A composition according to claim 35, wherein the at least one filler is present in an amount ranging from 0.01% to 60% by weight, relative to the total weight of the composition.

37. A composition according to claim 36, wherein the at least one filler is present in an amount ranging from 1% to 50% by weight, relative to the total weight of the composition.

38. A composition according to claim 1, wherein the composition comprises at least one cosmetic ingredient chosen from vitamins, thickeners, lipophilic or hydrophilic gelling agents, trace elements, softeners, sequestrants, fragrances, acidifying or basifying agents, preserving agents, sunscreens, surfactants, antioxidants and fibers, or mixtures thereof.

39. A cosmetic composition according to claim 1, wherein the composition is in the form of a suspension, a dispersion, a solution, a gel, an emulsion, a multiple emulsion, a cream, a paste, a mousse, a dispersion of vesicles, a two-phase or multiphase lotion, a spray, a powder, a paste, a stick and a cast solid.

40. A cosmetic composition according to claim 39, wherein said emulsion is chosen from oil-in-water emulsions (O/W) and water-in-oil (W/O) emulsions.

41. A cosmetic composition according to claim 39, wherein said multiple emulsion is chosen from water-in-oil-in-water emulsions (W/O/W), polyol-in-oil-in-water emulsions (polyol/O/W) and oil-in-water-in-oil (O/W/O) emulsions.

42. A composition according to claim 1, wherein the composition is in the form of a loose, compacted or cast powder.

43. A composition according to claim 1, wherein the composition is in a form chosen from a makeup rouge, an eyeshadow, a makeup product for keratin fibers, a mascara, and an eyeliner.

44. A process for obtaining a makeup film with a metallic and/or glossy effect on keratin materials comprising applying to said materials at least one composition comprising, in a cosmetically acceptable medium, a fatty phase and at least 30% of at least one first dyestuff comprising at least one metallic substrate coated with at least one layer of at least one mineral material.

45. A cosmetic process for making up keratin materials, comprising applying to said keratin materials at least one composition comprising, in a cosmetically acceptable medium, a fatty phase and at least 30% of at least one first dyestuff comprising at least one metallic substrate coated with at least one layer of at least one mineral material.
